(19) United States
(12) Plant Patent Application Publication
(10) Pub. No.: US 2009/0165180 P1
(43) Pub. Date: Jun. 25, 2009

(54) Cuphea plant named 'Cuped'

(50) ;

(75) Inventor: Henricus G.W. Stemkens, Andijk (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 12/002,824

(22) Filed: Dec. 19, 2007

(51) Int. Cl. A01H 5/00

(52) U.S. Cl. PLT/420

(57) ABSTRACT

A new Cuphea plant, particularly distinguished by its deep red flower color and compact plant habit.

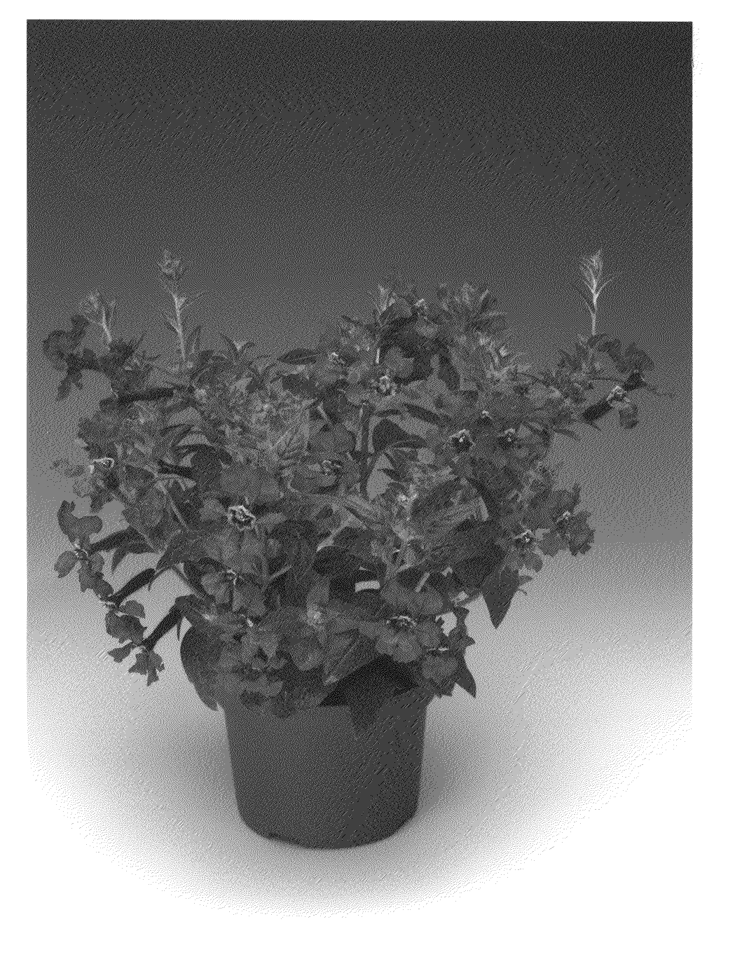

### LATIN NAME OF THE GENUS AND SPECIES OF THE PLANT CLAIMED

Cuphea hybrida

### VARIETAL DENOMINATION

‘Cuped’

### BACKGROUND OF THE NEW PLANT

The present invention comprises a new distinct cultivar of Cuphea, botanically known as Cuphea hybrida.

The new Cuphea is a product of a planned breeding program conducted in Enkhuizen, Netherlands. The new Cuphea cultivar is early flowering, has a compact plant habit, and a deep red flower color.

The new Cuphea is propagated by cuttings resulting from a single seedling. This seedling originates from a selection in Cuphea ‘D0369-1’ (not patented) pollinated with an unknown male parent. Seeds of this cross were sown in spring 2003 in Enkhuizen, Netherlands. The seedling was selected in August 2003 in Enkhuizen.

The present cultivar has been repeatedly asexually reproduced by cuttings in Enkhuizen, Netherlands, since 2003. The distinctive characteristics of this new Cuphea are stable and reproduced true to type in successive generations of asexual reproduction.

The new Cuphea plant is a perennial in zones 8 to 9 in the US.

### DESCRIPTION OF THE DRAWING

The new Cuphea plant is illustrated by the accompanying photographic drawing which shows blooms, buds and foliage of the plant in full color, the color showing being as true as can be reasonably obtained by conventional photographic procedures.

### BOTANICAL DESCRIPTION OF THE PLANT

The following detailed descriptions set forth the distinctive characteristics of this new Cuphea. The data which defines these characteristics were collected from asexual reproductions carried out in Enkhuizen, Netherlands. The plant history was taken from 13 week old plants. The instant plant was grown in an 11 cm container in an outdoor container field.

Color references are primarily to the R.H.S. Colour Chart of The Royal Horticultural Society of London.

Plants of the new Cuphea differ primarily from the plants of the originator ‘D0369-1’ in the following characteristics:

Flowers of the new variety are deep red colored whereas plants of the originator are rose colored. Plants of the new variety flower very early whereas plants of the originator flower late. Flowers of the new variety are bigger than the flowers of the originator.

TABLE 1
DIFFERENCES BETWEEN THE NEW CULTIVAR
‘CUPED’ AND A SIMILAR CULTIVAR

| | | ‘Flamenco Rumba’ (U.S. Plant |
|---|---|---|
| | ‘Cuped’ | Pat. No. 17,213) |
| Plant height | 20 cm | 40 cm |
| Flower size | 26 mm | 22 mm |
| Plant width | 20 cm | 30 cm |

The Plant:
   Classification—botanical.—Cuphea hybrida.
Parentage:
   Originator selection.—‘D0369-1’ (Not patented).
Propagation:
   Type of cutting.—Terminal cuttings.
   Time to initiate roots.—6-12 days at 18° C.
   Time to produce a rooted cutting.—10 days.
   Root description.—Fine, fibrous and light yellow in color.
Plant description:
   Form.—Globular, symmetrical, mounded.
   Height.—20 cm.
   Plant diameter.—24 cm.
   Branching habit.—Freely basal branching.
The stem:
   Shape.—Round.
   Color.—137B.
   Texture.—Pubescent.
   Length.—15-20 cm.
   Diameter.—4-6 mm.
   Internode length.—20-30 mm.
   Quantity.—Approximately 25 per plant.
The foliage:
   Type.—Simple.
   Arrangement.—Alternate.
   Number of leaves per stem.—15-25.
   Size of leaf.—Length: 18 mm Width: 10 mm.
   Shape of leaf.—Elliptic.
   Shape of apex.—Acute.
   Shape of base.—Obtuse.
   Texture.—Pubescent.
   Margin.—Entire.
   Venation.—Pinnate.
   Fragrance.—Present, musky.
   Color.—Upper surface: 137B Lower surface: 137D.
   Petiole.—Absent.
The flower:
   Natural flowering season.—From April until October.
   Inflorescences.—Spike.
   Form.—Close to salverform.
   Appearance.—Single flowers.
   Aspect.—Facing upward or outward.
   Flower size.—Diameter: 4 mm Depth: 16 mm.
   Disc diameter.—18 mm.
   Number of flowers per plant.—360.
   Flower opening/closing behavior.—Flowers stay open permanently.
   Fragrance.—Present.
   Bud (just before flowering).—Shape: Oval Diameter: 2 mm Length: 4 mm Color: 138B Texture: Pubescent.
   Pedicel.—Color: 138B Shape: Cylindrical Length: 3-6 mm Diameter: 1 mm Texture: Pubescent.
   Corolla tube.—Color (ventral surfaces): 45B Color (dorsal surface): 45A Tube shape: Tubular Tube apex: Flared Tube base: Cuneate Petals: Apetalous Calyx: Absent Valvate lobe number: 6 sub-equal valvate lobes, 4 large and 2 small Valvate lobes attachment: Individual valvate lobes are attached at summit of hypanthium. Large valvate lobe dimensions: 9 mm in width and 11 mm in length Small valvate lobe dimensions: 2 mm in width and 4 mm in length Valvate lobe color: 45B Valvate lobe texture: Smooth Valvate lobe appearance (adaxial and abaxial surfaces): Irridescent Valvate lobe shape: Rotund, unfused Valvate lobe apex: Obtuse Valvate lobe base: Cuneate and rounded Valvate lobe margin: Entire.
   Lasting quality of flowers.—On the plant: 3 days Cut flowers: 10 hours.
Reproductive organs:
   Androecium.—Stamens quantity: 8 Anther shape: Ovoid Anther length: 2 mm Anther color: 144C Pollen amount: No pollen.
   Gynoecium.—Pistil quantity: 1 Pistil length :1 mm Stigma shape: Bi-lobed Stigma color: 144C Style length: 14 mm Style color: 144D Ovary color: 144C.
Seed development: No seed set has been observed to date.
Disease resistance: No disease/pest resistance has been observed to date.
Weather tolerance: Heat tolerant to 42° C.; susceptible to wet conditions.

What is claimed is:

1. A new and distinct cultivar of Cuphea plant named ‘Cuped’, as substantially illustrated and described herein.
